(19) United States
(12) Patent Application Publication
(10) Pub. No.: US 2010/0049756 A1
(43) Pub. Date: Feb. 25, 2010

(54) Medical Intelligence Framework

(75) Inventors: Vamsi K. Chemitiganti, Coatesville, PA (US); Lucian Vlad Lita, San Jose, CA (US); Maleeha Qazi, King of Prussia, PA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA. Inc., Malvern, PA (US)

(21) Appl. No.: 12/258,758

(22) Filed: Oct. 27, 2008

Related U.S. Application Data
(60) Provisional application No. 60/984,469, filed on Nov. 1, 2007.
(60) Provisional application No. 60/984,482, filed on Nov. 1, 2007.
(60) Provisional application No. 60/988,902, filed on Nov. 19, 2007.
(60) Provisional application No. 60/988,893, filed on Nov. 19, 2007.

(51) Int. Cl. G06F 17/30; G06F 3/048

(52) U.S. Cl. 707/600; 707/E17.009; 715/764

(57) ABSTRACT

A framework executing on a computational structure and supporting a plurality of simultaneously executing software applications with a shared layer, wherein the framework is disposed between the plurality of applications and a set of data sources, the framework decomposing, processing, and analyzing data passed between the plurality of applications and the data sources into information elements.

### CROSS-REFERENCE TO RELATED APPLICATIONS

This application claims the benefit of Provisional Application No. 60/984,469 filed on Nov. 1, 2007, Provisional Application No. 60/984,482 filed on Nov. 1, 2007, Provisional Application No. 60/988,902 filed on Nov. 19, 2007, and Provisional Application No. 60/988,893 filed on Nov. 19, 2007, each filed in the United States Patent and Trademark Office, the contents of which are herein incorporated by reference in their entirety.

### BACKGROUND

1. Technical Field

The present invention relates to a data-mining, processing, and monitoring framework, and more particularly to a framework for support of an online, parallel set of processes that monitor, process and analyze data sources and data flow.

2. Discussion of Related Art

Hospitals face a supply chain problem in regards to managing inventory of costly medicines and surgical tools. One of the supply chain management bottlenecks in healthcare is the lack of agility; that is, being able to supply institutions with a required volume of items, products and equipment as they are needed. Most healthcare institutions employ complex data-acquisition and processing systems that typically span multiple modalities and multiple input types and sources. These existing practices make it time-consuming to acquire and synthesize information about item usage, stock levels and future needs (e.g., raw materials, medications, medical tools, supplies).

There are a number of clinical information systems in place that provide information to achieve a higher level of safety in certain situations. These systems include computerized results, the notes of nurses and other physicians, ordering tracking systems, patient tracking systems, scheduling systems, e-mail and other message systems, computerized clinical reference sources (e.g., drug pharmacopoeias, online clinical texts and journals, clinical protocols and guidelines), event monitoring systems, and other computerized decision support systems.

One issue that surgical departments, intensive care units (ICUs) and emergency departments (EDs) in hospitals face is that of medical errors in the therapy and treatment of patients. This is further complicated by basic errors, which can be more likely to happen if side effects are delayed or unpredictable, if there is a longer survival or latent interval, or if a patient has been transferred from one facility to another. Medical errors can include errors made in the treatment of patients, medication errors and deviations from standard practice methodologies.

Therefore, a need exists for a system that can mine data for patients as data is entered, observe trends in diseases, observe trends in resource usage, notify medical staff of abnormal patient signal levels, flag medical errors online and offline, and support remote assistance by experts.

### BRIEF SUMMARY

According to an embodiment of the present disclosure, a framework executing on a computational structure and supporting a plurality of simultaneously executing software applications with a shared layer, wherein the framework is disposed between the plurality of applications and a set of data sources, the framework decomposing, processing, and analyzing data passed between the plurality of applications and the data sources into information elements.

The framework may performs shared parallel decomposition, processing, and analysis of the data into information elements for two or more applications of the plurality of software applications.

The shared layer is one of a listener, a monitor, a tracker, and an action trigger processing the information elements.

The data sources may serve the data, wherein the data may be extracted from multiple modalities and the framework aggregates the information elements decomposed from the data.

The framework may aggregate the information elements decomposed from the data and the shared layer interprets the information elements to determine a trend.

The framework may perform data extraction, data processing, and data mining for support of a decision support application. The extraction may include extracting the information elements by means of natural-language processing of free text and by means of data mining from discrete data fields.

The action trigger may perform a prediction about future data based on the relevant information elements.

Filtering is performed on the information elements to look for conditions indicative of a trend. The information elements may be filtered to identify a new trend.

The framework may be connected to a central gateway, wherein the central gateway allows data to be exchanged between the framework and another framework.

The framework can include a user interface, wherein the user interface allows users to manipulate the elements.

The action trigger may be one of a predefined trigger or a user-defined trigger.

According to an embodiment of the present disclosure, a computer readable medium is provided embodying instructions executable by a processor to perform a method of monitoring and processing data. The method includes executing, simultaneously, a plurality of software applications with a shared component, and decomposing data passed through the shared layer into an element accessible by the plurality of software applications.

The framework may perform shared parallel decomposition of the data into information elements for two or more applications of the plurality of software applications. The shared layer may be one of a listener, a monitor, a tracker, and an action trigger processing the elements.

The data resource may serve the data, wherein the data may be extracted from multiple modalities and the framework aggregates the information elements decomposed from the data.

### BRIEF DESCRIPTION OF THE DRAWINGS

Preferred embodiments of the present disclosure will be described below in more detail, with reference to the accompanying drawings:

FIG. 1 is illustrates a framework supporting applications according to an embodiment of the present disclosure;

FIG. 2 is a flow diagram showing data handling between an application and resource according to an embodiment of the present disclosure;

FIG. 3 is a flow diagram of a method of data decomposition into information elements according to an embodiment of the present disclosure; and

FIG. 4 is a diagram of a computer system for implementing a support framework according to an embodiment of the present disclosure.

### DETAILED DESCRIPTION OF PREFERRED EMBODIMENTS

According to an embodiment of the present disclosure, a intelligence framework (hereinafter “framework”) supports data-mining, processing, monitoring, etc. The framework supports an online, parallel (e.g., shadow) set of processes that monitor and process data sources and data flow. The framework does not interfere or directly affect institution information systems.

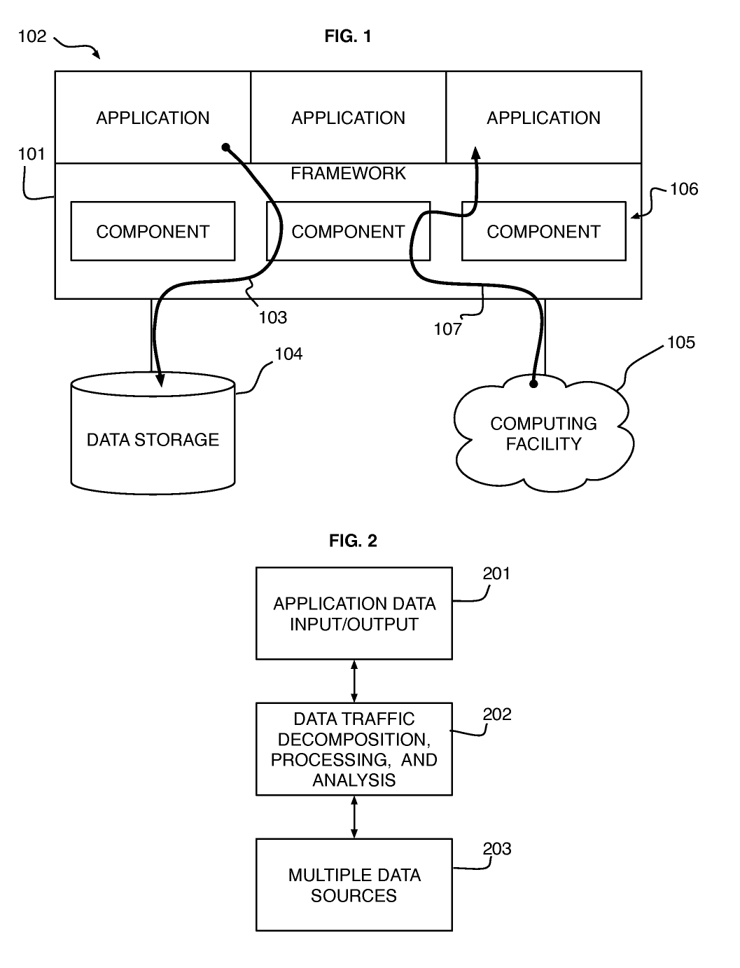

Referring to FIG. 1, the framework 101 can be implemented in various applications 102 for continuous online monitoring and processing of data and data traffic, e.g., 103, to and from resources such as a data storage 104 or computing facility 105. The computing facility 105 may include, for example, a grid computing environment, a cloud computing environment, set of servers, etc. Further the computing facility 105 may include another framework, a gateway for connecting to another framework, etc. Rather than performing redundant, non-standard data extraction and processing, the framework 101 provides the common basis for the applications 102. Further, structured and unstructured data (e.g., demographics, reports, order information) are monitored and mined, and the data is decomposed into information elements and continuously indexed online, for example, storing the information elements on the data storage 104.

The information elements may be, for example, specific key terms or phrases extracted from the data flow. The existence of a word or phrase may be used to show the existence of the state of the patient. The existence of the word or phrase may be used with other information to infer a state. Rules may be used to determine the contribution of any identified word to an overall inference. Certain conditions may be implied through a reference to related symptoms or diseases and never mentioned in the data flow explicitly. Information extraction may include a combination between hidden Markov models and language modeling approaches for named entity extraction, conditional random fields for sequence data labeling in general English text, and biomedical text.

The framework executes on a computational structure including, for example, one or more processing units, networking, and storage. The framework 101 can be implemented with components 106 for providing different services; the components 106 represent a shared layer of the framework 101. The framework 101 supports the construction, integration and customization of different components 106. The components 106 include, for example, listeners, event monitors, aggregation trackers, and action triggers. The listeners generate alerts based on the available static data and data traffic (e.g., disease outbreaks monitoring). The event monitors detect, for example, admissions, discharges, report transcriptions, etc. The aggregation trackers are used for collecting information about resource consumption and stock levels. The action triggers generate and propagate orders based on listeners, monitors, and trackers and perform decision support for contra-indications, suggestions, chart generators etc. The components 106 may pass the data among each other, e.g., 107, to perform complex processes, e.g., data aggregation and trend spotting or listening and monitoring data, under the direction of the framework 101 without affecting the applications 102.

Furthermore, the framework 101 supports various applications 102, including supply chain management (SCM), natural language processing (NLP), enabled medication intelligence systems, and surgical monitors. The framework 101 is not limited to these applications.

Referring to FIG. 2, the applications send and receive data 201 utilizing the same continuous monitoring and processing of data and data flow by sharing components of the framework, wherein data traffic is handled by the framework. The data traffic is decomposed, processed, and indexed 202 by the framework; the framework is in a position to eliminate redundant processes for multiple applications in a standard way. Similarly, data traffic to and from the resource 203 is decomposed and indexed 202. Structured and unstructured data (demographics, reports, order information, etc.) is monitored and mined—the data is decomposed into information elements and continuously indexed by the framework for processing by the components.

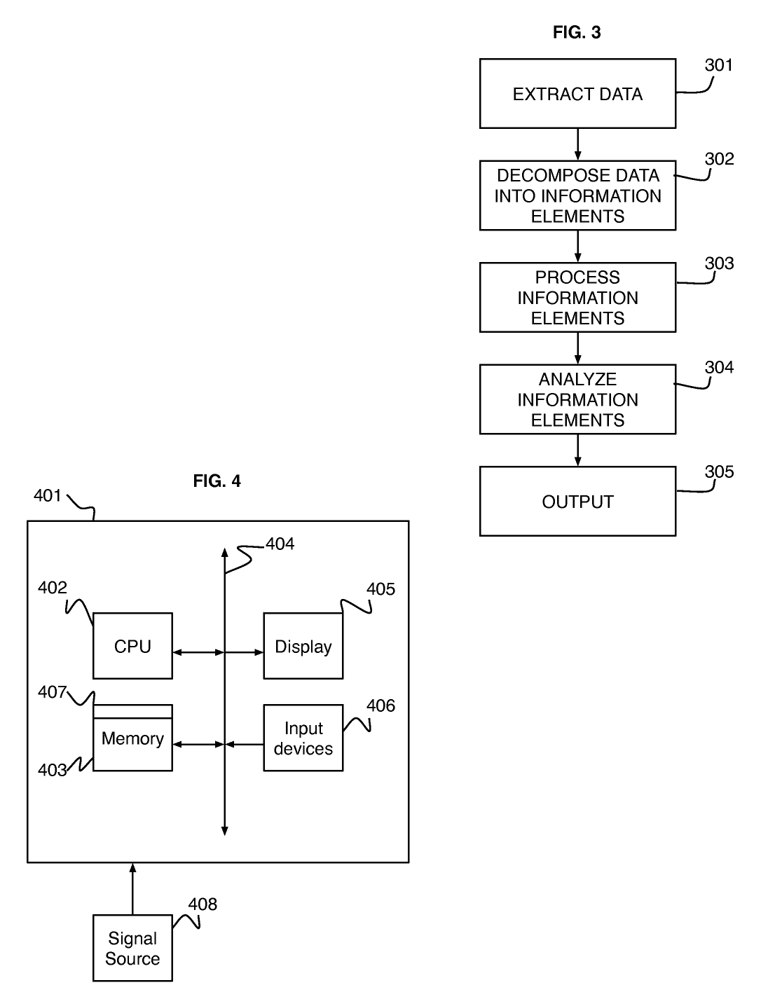

Referring to FIG. 3, data is extracted from one or more data sources 301, which may have different modalities. The data sources can include patient monitoring systems, physician and nurse data entries, admission forms, medical resource levels, etc. The extracted data is decomposed into information elements 302. The information elements may be processed by the components and/or applications 303, for example, indexing the data elements. The information elements can be analyzed 304 to produce an outcome 305 (e.g. visualization, decision support, warnings etc). The information elements may be entities; for example, a semi-supervised method may be used identify complex medical entities (e.g., medication, diseases, symptoms, or others) that include relevant modifiers, compound structures, and paraphrases. The entities may be identified from electronic patient records, along with building an extended medical class lexicon. The exemplary semi-supervised approach extracts extended entities from free medical text, such as noisy patient records, using single or a few initial terms. A large, domain specific set of entities can be extracted starting from different sized existing knowledge sources. The extraction process, which may be performed automatically without human involvement, incrementally incorporating new elements.

Data driven approaches may automatically discover new information elements of a concept, based on, for example, co-occurrence and context similarity assumptions. Members of medical concepts such as symptoms, medications, diseases, and medical tests are automatically extracted from the data flow through the framework and indexed as information elements; the information elements are high-level data as compared to the data flow, and include, for example, filtered words and phrases indexed into concepts, which may be analyzed by the components of the shared layer and/or the applications.

One exemplary application is a supply chain management (SCM) specific application. The SCM is a pro-active application of data/text mining under which information extraction from structured and unstructured data can provide pro-active statistics about resource consumption, flag needs, and pre-update stock levels. Using the pro-active statistics, the SCM may, for example, preemptively propagate orders.

According to an embodiment of the present invention, the SCM application may utilize the framework to manage inventory. The SCM application is a POCKET (Point of Consumption Knowledge Extraction and Tracking) application. POCKET is an information based method that allows medical institutions to streamline the process of inventory management and improve cost optimization from a supply chain management perspective.

POCKET is supported by the framework to perform data extraction, data processing and basic data mining. Data mining and natural-language based information extraction are performed at the point of consumption. This acquired information is used to drive supply chain logistics. The framework is used by POCKET to set up listeners and aggregation trackers for extracting focused information (e.g., procedures performed, resources used, item reusability, stock levels) from multi-modality input sources at the point of consumption. Many of these input sources are structured or semi-structured (e.g., electronic forms, inventory updates in tabular forms), while others may include noisy, free text inputs (e.g., typed free text reports, transcribed notes, scanned documents). This focused information is aggregated at a global level. According to an exemplary embodiment of the present disclosure, the extraction and aggregation can be performed substantially in real-time, allowing medical institutions to plan ahead, better define their needs, reduce costs and lower the risk of not having essential medicines and surgical tools available when needed. Based on the extracted focused information, POCKET implements the framework to set up action triggers that perform optimization and prediction algorithms that can forward assess inventory needs, balance supply and demand, and notify those responsible for managing inventory so that appropriate action can be taken when needed.

POCKET can help with demand forecasting in the healthcare supply chain by reading free text, applying natural-language processing (NLP; NLP includes a set of automated techniques that convert narrative documents into a format that allows for computer based analysis) techniques, filtering out noise and helping the forecaster interpret current stock levels. According to an embodiment of the present disclosure, POCKET can also integrate with applications like SAP APO (Advanced Planner and Optimizer) to help predict demand for supplies across multiple hospitals that comprise a larger health system. POCKET can also be utilized to analyze patient data in specialties like cardiology, oncology and critical care to alert administrators when there are low levels of critical supplies and medicines, and current stock levels can then be compared with available stocks. If the information related to the inventories of medical institutions is rapidly propagated, the needs of the institutions can be defined instantaneously at the enterprise level. Based on the up-to-date, overall aggregated information, decisions can be made much quicker and action can be taken rapidly to ensure the inventory is managed efficiently.

Another exemplary application is a Medical Trend Manager (MTM); a Natural Language Processing (NLP) MTM is a shadow process that aggregates and interprets data to spot outbreaks, medical trends, provide online support to local policy makers and raise alarms when monitoring mechanisms have been set. For example, in a hospital setting the use of remote transcription services for processing medical notes and diagnoses requires that data be sent via the Internet. The data may be intercepted for handling by the framework to, for example, increase efficiency and effectiveness of operations.

The interpretation of the information elements can be done at the shared layer level to determine trends and perform the processing, as well as at the application level, wherein individual applications perform additional analysis and processing for more specific goals.

According to an embodiment of the present disclosure, an application utilizes the framework to implement NLP techniques designed to mine medical transcription data across hospitals within a specific geographic area in order to look for trends in fast breaking infections and outbreaks (e.g., staphylococcus, bird flu). This application, referred to hereinafter as MTM (Medical Trend Manager), detects trends that occur in a localized pattern and notifies the CDC and other appropriate authorities.

MTM is supported by the framework to perform data extraction, data processing, and basic data mining. MTM uses the framework to set up listeners and monitors, based on incoming patient data, in order to track various disease outbreaks, statistical trends and anomalies. The MTM implements the framework to set up action triggers that notify medical institutions, the CDC, and other appropriate authorities of trends in fast breaking infections and outbreaks. MTM may be implemented as a stand-alone trend spotting service provided to hospitals that choose to sign up for it. This service can also provide appropriate security and data privacy to the hospitals. MTM may also be implemented to utilize existing commercial medical transcription systems provided by third party companies. Under this implementation, all data is already safe via an existing established data transfer security system used by the third party companies. As information flows out of a hospital en route to these third party companies, MTM monitors this information by implementing the listeners provided by the framework. In a preferred embodiment, multiple systems may be installed in various hospitals and information based on the information elements may be exchanged between these hospitals using a central gateway. Using the framework, MTM can filter out vast amounts of unneeded data and noise to arrive at an accurate understanding of the correct trends. Specifically, MTM can use the framework to perform text normalization across data sources (e.g., the hospitals) and adapt extraction models, which are based on corresponding entities and events, such that only new trends are identified. For instance, instead of extracting solid information useful in a day-to-day clinical environment, MTM focuses on extracting initial outliers that consolidate over time, thus forming new trends. MTM considers background information to be normal entity and event distributions, and this background information is monitored over long periods of time. In order to filter background events, MTM may continuously compile statistical information of natural language from transcription records.

According to an embodiment of the present disclosure, a secure web portal and a user interface are provided to enable disease experts to specify the type of information they are searching for (e.g., anthrax outbreak and symptoms in a given geographic area). Constrained natural language querying capabilities are provided, as well as an aggregate text exploration user interface, which facilitates rapid trend identification and labeling. Expert user feedback can also be incorporated to provide more accurate predictive power. MTM can also be utilized to detect naturally occurring conditions so that action can be taken; known periodic or episodic naturally occurring conditions can be profiled by expert users, and listeners can be implemented to analyze the processed data stream and look for specific event types. Substantially real-time results are combined into an easy to view and secure web portal. MTM may also be implemented to set up alerts based on specific keywords and their combined meanings, and personnel may be contacted via e-mail, cellular phones, pagers, and other available communication means. An archive of historical information can also be maintained for comparison purposes.

MTM can be adapted to utilize NLP extraction techniques such as semantic analysis, parsing and event extraction to mine vast databases of information. Rather than implementing statistical NLP algorithms which implement time-consuming optimization methods, faster, but lower precision algorithms are normally used. However, higher precision algorithms may be implemented on data stream portions which exhibit potentially unusual patterns, thus contributing to the scalability aspect of the platform. MTM may also be used to generate metrics for policymakers.

A further exemplary application is an NLP Enabled Medication Intelligence (NEMI); an NLP Enabled Medication Intelligence System acts as a parallel narrative document processing process that is able to provide decision support (suggestions, contra-indication spotting, history and patient data synthesis).

According to an embodiment of the present disclosure, an application utilizes the framework to integrate with commercial medication ordering systems. For example, the framework may process information including current and past medications prescribed to a patient, a patient's past conditions and illnesses, patient specific details, drug specific details and new studies. This application, referred to hereinafter as NEMI (NLP Enabled Medication Intelligence), may act as both a medication conflict detecting application and an online structured and unstructured data monitoring and mining application. Based on an explicit and implicit flow of information, NEMI can implement both predefined triggers (e.g., contra-indications) and user-defined triggers (e.g., symptom warning, patient education tips, rare but existing risks) when appropriate.

NEMI implements the framework to perform data extraction, data processing, and data mining, which includes data obtained from discharge summaries, order information and patient history. NEMI uses the framework to set up listeners and monitors for incoming orders, patient data, and discharge events. NEMI uses the framework to set up action triggers for contra-indications and order submissions.

The information extraction and filtering component of NEMI can interpret a patient specific timeline, which may include partial and potentially noisy information regarding medications prescribed in the past, potential illnesses and conditions a patient has or may have had in the past. Using this information, specific events can be triggered and then handled according to the specification of a physician. The action to be taken, as well as the confidence thresholds involved, can be user-specific, thus reducing the number of false negatives (false alarms) while still covering most problematic situations. The information processing can be performed either online by the framework while the medical professional is writing the prescription, or offline before the order is actually placed by the medical professional. Online processing requires additional NLP extraction components. For instance, depending on the input method, online speech-to-text conversion might be required. Further, hard constraints are imposed on the processing time so that the response time of the alert system is kept low, thus allowing the physician to take corrective action before it is too late. Online processing also results in concurrent order processing. For example, as the order is entered (e.g., by dictation or typing), NEMI can immediately start filtering and extracting relevant information before the user finalizes the order. This may result in a faster response time and may also allow alerts to come into focus before an order is actually placed.

NEMI may also allow for a prescription to be regenerated and modified. This results in the overall time being reduced and allows the feedback loop to be integrated with the input method. Tight integration of the feedback loop with the input system ensures faster response time and makes it more likely that the user will maintain the current context (i.e., it is unlikely that the user will switch back and forth between different patients, potentially missing important patient record details). A statistical classifier or a reasoning method such as a decision tree or Bayes network can be used to perform reasoning. Reasoning results in the encoding of either the expert logic or the statistical mechanism for contra-indications related to specific drugs and conditions, and also results in compliance with standards and regulations (e.g., those set by the JCAHO). Pre-processing includes tokenization, speech tagging, shallow parsing (or parsing if the task is not performed online), and semantic parsing. Once the data is pre-processed, the system will extract named, nominal and pronominal entities, assess and label relations between entities, and extract specific roles that the entities play (e.g., physician, patient, referring physician). Based on the extracted information, new filters are trained to look for specific conditions (e.g., HF, AMI, pneumonia), potential contra-indications, and compliance problems.

NEMI keeps track of and learns how to extract new information about specific patients and specific drugs. NEMI also encodes the logic of new studies. Although the system can be maintained by a human, automatic updates can be triggered by the latest guidelines. The statistical NLP models used in NEMI allow for incremental training such that new data can be analyzed and incorporated over time.

According to an embodiment of the present disclosure, an application, referred to hereinafter as SM (Surgical Monitor), utilizes the framework to help surgical departments prevent avoidable errors in real-time. This is accomplished by integrating the application with commercial surgical electronic health record systems.

The SM implements the framework to perform data extraction, data processing, and basic data mining, which includes data obtained from transfer notes, surgery reports and patient history. SM uses the framework to set up listeners and monitors for surgery events, reports, transfers and discharge. SM utilizes the framework to set up action triggers to flag errors, potential side effects and possible alternate treatments.

SM implements the framework to automatically detect relevant data from the ICU, ED and EHR databases regarding patients who are either in the process of being operated on or may be operated on. This is accomplished using NLP processing in conjunction with patient notes, assessments, history and physicals, consultation notes and lab reports. Relevant information may also be processed from multiple clinical data sources in real-time as they are entered (e.g., notes, patient records, ADT system, financial system, surgery records). Results can be combined into an easy to view and secure web portal accessible by physicians and administrators throughout the enterprise. This allows senior physicians to remotely supervise junior surgeons performing complex, but urgent surgery.

SM allows contradictions between existing protocols and currently administered procedures to be identified. Once identified, the physician can determine whether the difference in procedure is desired or whether it is indicative of a potential problem. The aggregate of these contradictions over time can provide a basis for the re-analysis of protocols. This information can be used by protocol creation agencies to ensure that guidelines are kept up to date.

SM can also be used to check for patient identity mistakes using more than just the bar codes currently used for identification. Thus, SM can help reduce the number of mistakes related to the wrong site of a surgery, the wrong person being operated on, the wrong procedure being performed and the wrong medications administered. For example, biometrics (e.g., retinal scan, voice recognition, fingerprints) as well as the detection of abnormal variations in a person's vitals statistics within a short period of time (e.g., heart rate monitor, BP monitor, O2 monitor, brain waves monitor) can be used for identification purposes. SM can also be integrated with input from specific hardware to detect anesthesia awareness during surgery, to detect whether a surgical team has the correct qualifications to perform a desired surgery, and to keep a record of all devices implanted in an individual, thus allowing a notification to be made to affected individuals in a timely fashion when problems with certain devices are discovered.

It is to be understood that embodiments of the present disclosure may be implemented in various forms of hardware, software, firmware, special purpose processors, or a combination thereof. In one embodiment, a software application program is tangibly embodied on a program storage device. The application program may be uploaded to, and executed by, a machine comprising any suitable architecture.

Referring now to FIG. 4, according to an embodiment of the present disclosure, a computer system 401 for supporting shared data traffic handling includes, inter alia, a central processing unit (CPU) 402, a memory 403 and an input/output (I/O) interface 404. The computer system 401 is generally coupled through the I/O interface 404 to a display 405 and various input devices 406 such as a mouse and keyboard. The support circuits can include circuits such as cache, power supplies, clock circuits, and a communications bus. The memory 403 can include random access memory (RAM), read only memory (ROM), disk drive, tape drive, etc., or a combination thereof. The present invention can be implemented as a routine 407 that is stored in memory 403 and executed by the CPU 402 to process the signal from the signal source 408. As such, the computer system 401 is a general purpose computer system that becomes a specific purpose computer system when executing the routine 407 of the present disclosure.

The computer platform 401 also includes an operating system and micro instruction code. The various processes and functions described herein may either be part of the micro instruction code or part of the application program (or a combination thereof) which is executed via the operating system. In addition, various other peripheral devices may be connected to the computer platform such as an additional data storage device and a printing device.

It is to be further understood that, because some of the constituent system components and method steps depicted in the accompanying figures may be implemented in software, the actual connections between the system components (or the process steps) may differ depending upon the manner in which the system is programmed. Given the teachings of the present disclosure provided herein, one of ordinary skill in the related art will be able to contemplate these and similar implementations or configurations of the present disclosure.

Having described embodiments for supporting shared data traffic handling, it is noted that modifications and variations can be made by persons skilled in the art in light of the above teachings. It is therefore to be understood that changes may be made in embodiments of the present disclosure that are within the scope and spirit thereof.

What is claimed is:

1. A framework executing on a computational structure and supporting a plurality of simultaneously executing software applications with a shared layer, wherein the framework is disposed between the plurality of applications and a set of data sources, the framework decomposing, processing, and analyzing data passed between the plurality of applications and the data sources into information elements.

2. The framework of claim 1, wherein the framework performs shared parallel decomposition, processing, and analysis of the data into information elements for two or more applications of the plurality of software applications.

3. The framework of claim 1, wherein the shared layer is one of a listener, a monitor, a tracker, and an action trigger processing the information elements.

4. The framework of claim 1, wherein the data sources serve the data, wherein the data is extracted from multiple modalities and the framework aggregates the information elements decomposed from the data.

5. The framework of claim 1, wherein the framework aggregates the information elements decomposed from the data and the shared layer interprets the information elements to determine a trend.

6. The framework of claim 1, wherein the framework performs data extraction, data processing, and data mining for support of a decision support application.

7. The framework of claim 6, wherein the data extraction includes extracting the information elements by means of natural-language processing of free text and by means of data mining from discrete data fields.

8. The framework of claim 3, wherein the action trigger performs a prediction about future data based on the relevant information elements.

9. The framework of claim 1, wherein filtering is performed on the information elements to look for conditions indicative of a trend.

10. The framework of claim 9, wherein the information elements are filtered to identify a new trend.

11. The framework of claim 1, wherein the framework is connected to a central gateway, wherein the central gateway allows data to be exchanged between the framework and another framework.

12. The framework of claim 1, further comprising a user interface, wherein the user interface allows users to manipulate the elements.

13. The framework of claim 3, wherein the action trigger is one of a predefined trigger or a user-defined trigger.

14. A computer readable medium embodying instructions executable by a processor to perform a method of monitoring and processing data, comprising the steps of:
executing, simultaneously, a plurality of software applications with a shared component; and
decomposing data passed through the shared layer into an element accessible by the plurality of software applications.

15. The computer readable medium of claim 14, wherein the framework performs shared parallel decomposition of the data into information elements for two or more applications of the plurality of software applications.

16. The computer readable medium of claim 14, wherein the shared layer is one of a listener, a monitor, a tracker, and an action trigger processing the elements.

17. The computer readable medium of claim 14, wherein the data resource serves the data, wherein the data is extracted from multiple modalities and the framework aggregates the information elements decomposed from the data.
